(19) United States
(12) Patent Application Publication
(10) Pub. No.: US 2010/0331777 A1
(43) Pub. Date: Dec. 30, 2010

(54) NOSE DEVICE

(75) Inventor: Per Danielsson, Ljungby (SE)

Correspondence Address: BROOKS KUSHMAN P.C., 1000 TOWN CENTER, TWENTY-SECOND FLOOR, SOUTHFIELD, MI, 48075, US

(73) Assignees: Goeran ALMGREN, Malmoe (SE); Per DANIELSSON, Ljungby (unknown)

(21) Appl. No.: 12/865,226

(22) PCT Filed: Jan. 29, 2009
(86) PCT No.: PCT/SE09/00060

§ 371 Date: Jul. 29, 2010

(30) Foreign Application Priority Data
Jan. 29, 2008 (SE) 0800216-4
Mar. 3, 2008 (SE) 0800746-0

(51) Int. Cl. A61M 29/02

(52) U.S. Cl. 604/104; 606/199

(57) ABSTRACT

The nose device has a conical portion with a distal end intended to be arranged adjacent a nose bone and a proximal end intended to be arranged adjacent a nostril. A screw winding is arranged centrally at the outer surface of the conical portion. A tab portion is arranged at the proximal end of the conical device. The nose device is inserted in the nose of the person until the distal end is positioned adjacent the nose bone of the person. Then, the nose device is rotated so that the distal end is positioned adjacent the nose bone of the person. When the nose device is rotated, the screw windings cooperate with the walls of the nose channel in order to pull them slightly outwards and stretch them, in order to thereby keep the nose channels open. A filter may be arranged for filtering the inhaled air.

### AREA OF INVENTION

The present invention relates to a nose device intended to be inserted through the nostril into a nose of a user.

### BACKGROUND OF INVENTION

Many people in all ages suffer from narrow nasal channels and breathing difficulties. Such problems may be chronic and can be aggravated during stress and can also give problems at less demanding activities.

Colds and allergies can influence upon the mucose membranes of the nose and may induce a reduced airflow through the nose, a stopped-up nose.

Many male persons have problems with snoring and sleep disorders, which may result in sleep apnea.

Narrow nose channels of athletes, which perform exercises and/or contests, can result in decreased results, at training as well as at contests.

U.S. Pat. No. 2,569,743 discloses a device to promote nasal breathing. The device comprises a tube with is adapted to fit into each nostril and which is provided with a relatively large bore to afford an ample air passage for inhaling and exhaling which is frequently not provided due to deformed, defective of otherwise impaired nasal passages which are either so restricted or closed that normal breathing through the nose may be ordinarily accomplished only with difficulty and as a result mouth breathing while sleeping naturally occurs and which additionally causes snoring.

U.S. Pat. No. 5,718,224 describes a nose dilator, which may be used by athletes for preventing the outer wall tissue of a nose channel from drawing in during inhaling. The nose dilator comprises a spring member, which extends over the nose. A pad with an adhesive surface covers the spring member and extends around the spring member so that there is a peripheral space between the spring member and the pad member. The spring is arranged centrally in the pad. An adhesive between the spring member and the cushion covers the entire spring member at the surface cooperating with the pad. This nose dilator may be effective in maintaining the nose channels open and enlarged at normal condition, but are essentially inoperative if the nose mucose membrane is swollen.

U.S. Pat. No. 5,665,104 discloses a nose dilator comprising two molded, rigid and essentially cylindrical insert portions interconnected by a connection rib. Each insert portion is inserted in a vestibule of the nose in order to expand the area of the nostril, thereby enhancing breathing. Similar devices are described in U.S. Pat. Nos. 5,895,409 and 6,971,388. However, these devices can easily be dislodged and fall out of the nose, especially if they are not dimensioned in order to more or less exactly fit in the cavities of the nose of the person using it.

There is a need for improvements of a nose device.

### DISCLOSURE OF INVENTION

Thus, an object of the present invention is to cure, remedy or eliminate one or several of the above-mentioned flaws and disadvantages, singly or in any combination.

Thus, according to an aspect of the invention, there is provided a nose device intended to be inserted in a nose channel of a person, comprising a conical portion with a distal end intended to be arranged adjacent a nose bone, and a proximal end intended to be arranged adjacent a nostril, whereby the distal end is the small end of the conical portion, whereby screw windings are adapted centrally at the outer surface of the conical portion. A tab portion may be arranged at the proximal end of the conical device. A recess may be arranged at a side surface of the conical portion.

The screw windings may comprise ridges having an essentially rectangular cross-sectional shape.

In an embodiment, a filter device may be arranged covering the internal passage of the conical portion for filtering air passing through the conical portion. An elastic device may be arranged at the remote end of the conical portion. A medical agent can be arranged at the nose device, such as histamine or penicillin. A connection unit can be adapted for connection of a gas supply device.

In another embodiment, the nose device can comprise a cylindrical portion at the distal end with a shoulder for cooperation with a nose bone.

The nose device can be made of a plastic material, such as polycarbonate, polyurethane or polyvinylcloride.

### BRIEF DESCRIPTION OF THE DRAWINGS

Further objects, features and advantages of the invention will appear from the following detailed description of embodiments of the invention with reference to the drawings, in which:

FIG. 1 is a perspective view from the side of an embodiment of a nose device according to the invention.

FIG. 2 is an enlarged and partly cut perspective view of the embodiment of the nose device according to FIG. 1.

FIGS. 3 to 9 are cross-sectional views of other embodiments.

FIG. 10 is an end view of the embodiment according to FIG. 9.

FIG. 11 is a perspective view of another embodiment.

FIG. 12 is an end view of the embodiment according to FIG. 11.

FIG. 13 is a perspective view of a further embodiment.

FIG. 14 is a perspective view of a still further embodiment.

FIG. 15 is a cross-sectional view of the embodiment of FIG. 11.

FIG. 16 is a cross-sectional view of a yet further embodiment.

FIG. 17 is a cross-sectional view of a yet still further embodiment.

### DETAILED DESCRIPTION OF EMBODIMENTS

Below, several embodiments of the invention will be described with reference to the drawings. These embodiments are described in illustrating purpose in order to make possible to a skilled person to perform the invention and in order to disclose the best mode. However, such embodiments do not limit the invention. Moreover, other combinations are possible of the different features disclosed in the different embodiments with the scope of the invention.

FIG. 1 is a perspective view of a person 1 which intends to use the nose device 10 according to an embodiment of the invention. The nose device is inserted in the nose 2 through the nostril 3 as is shown by the arrow 4 so that the nose device 10 is adapted more or less complete inside the nose 2. Only a small flange portion 12 extends out from the nostril 3 in order to be gripped by the fingers. The nose device 10 is provided with an outer screw winding 11 as shown.

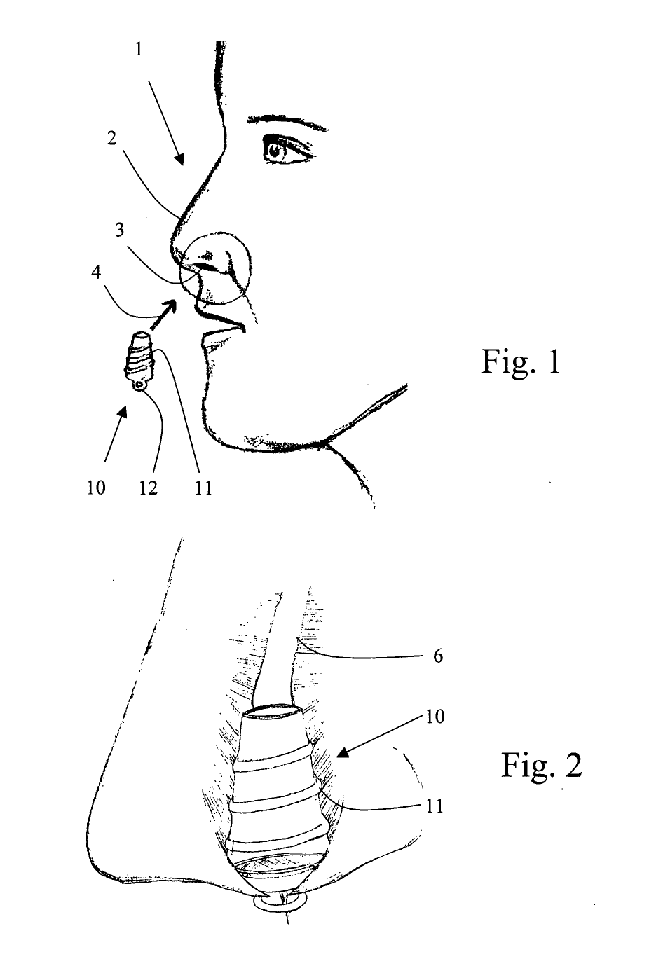

The nose device has a length which is dimensioned so that when the nose device is inserted in the nose, it reaches almost up to the outer end of the nose bone 5 of the person, see FIG. 2, wherein also the nose channel 6 is visible. The nose channel 6 is pleated or “warped” by conques in a way that differs between different persons. Moreover, the nose channel can be more or less obstructed due to swollen mucosa or due to the fact that slime is produced during an infection decease.

The nose device is inserted in the nostril and the nose channel 6 until the distal end of the nose device 10 is adjacent the nose bone 5. Then, the nose device 10 is rotated around its longitudinal axis over half a revolution or more, resulting in that the screw winding 11 cooperates with the opposite inner surface of the outer portion of the nose channel 6.

The torque exerted by the fingers at the flange 12 is converted to a rotating movement of the nose device. The rotating movement will normally result in that the nose device is screwed further into the nose channel. However, such a further screwing into the nose channel is prevented by the fact that the distal end of the nose device abuts towards the nose bone 5. Instead, the torque and the rotation will result in that the surfaces of the nose channel cooperating with the screw windings are screwed or streched downwards in FIG. 2, that is are extended, stretched of elongated in the longitudinal direction of the nose device. Such elongation of the side surfaces of the nose channel will give results also longer up in the nose channel above the nose device, so that also the side surfaces of the nose channel above the nose device are elongated to a certain extent. Such an elongation will result in that the nose channel will be kept open. In addition, a possible tendency of collapsing of the nose walls of the outer portion of the nose channel will be prevented by the structure of the nose device.

Experiments have shown that the above-described embodiment of a nose device is effective in providing open nose channels during conditions of swollen nose mucosa, such at a cold infection and allergic reactions.

The nose device is also effective in providing a large open nose channel for athletes desiring an improved air take-up or breathing ability. Persons suffering from sleep disorders will also have use of the nose device, since it keeps the nose channels open during sleep.

The nose device or the nose expander is constructed for expanding the nose channel by spreading the mucosa for increased airflow and in order to make possible improved breathing. The object of the nose expander is to expand the nose channel with a lightweight, inner expander, which due to its format and insertion function will extend or strech the nose walls and thus increase the airflow.

The nose expander comprises a conical cylinder with an outer screw winding. The pitch and the depth of the windings are construed in order to achieve a function, which is efficient and which leniently enlarges the air channels of the nose and at the same time stretches the mucosa of the walls of the nose channel in order to make easier the air flow at inspiration and expiration of air.

An advantage of the nose device is that it is securely arranged inside the nose channel due to the screw windings and due to the cooperation with the outer end of the nose bone. Thus, the nose device allows active body movements without being dislocated from its position, even at heavy athletic activities. Also movements during sleep will not dislocate the nose device.

Another advantage with the nose device is that it extends the inner surface of the nose channel opposite the nose device so that an expansion effect of the nose device is effective also further up along the nose channel.

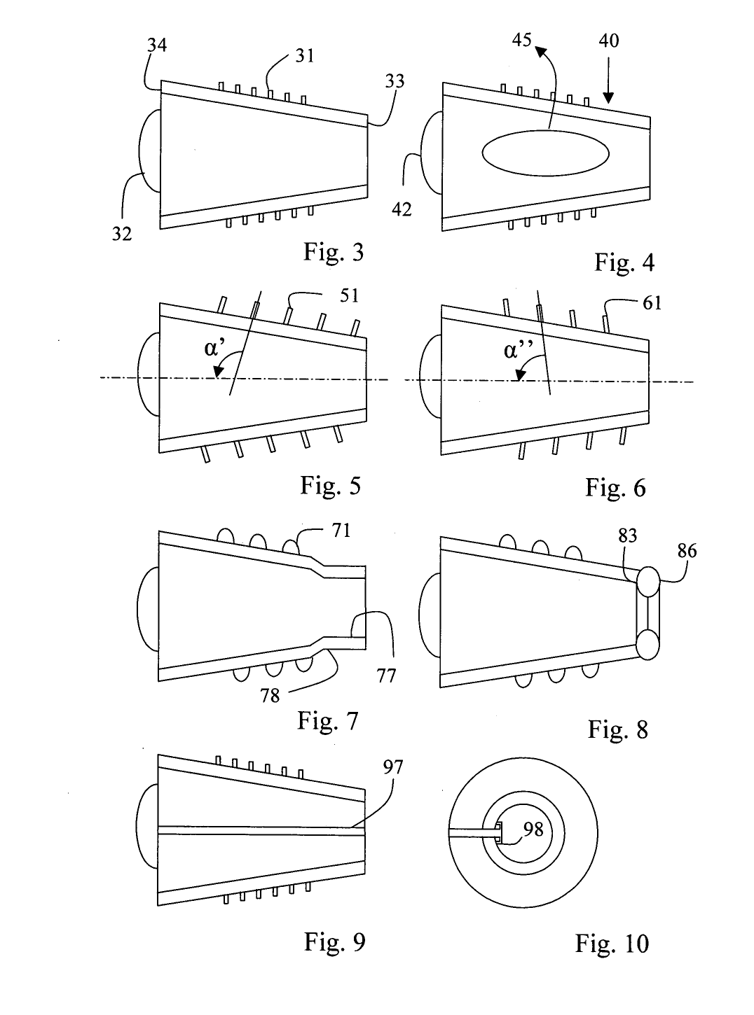

FIG. 3 is a cross-sectional view of another embodiment of the nose device.

The nose device 30 comprises a distal end 33 intended to be arranged adjacent the nose bone and a proximal end 34 intended to be arranged adjacent the nostril, i.e. the opening of the nose. The nose device is conical so that the proximal end is larger than the distal end.

In an embodiment, the cone angel is about 9°. The length of the nose device is about 25 mm. The diameter of the front end is about 18 mm and the diameter of the remote end is about 10 mm. The material thickness is about 1.5 mm.

A grip tab 32 is attached to the proximal end along a portion of its circumference, such as over about 90°. The tab has a length of about 3 mm in the longitudinal direction of the nose device.

A screw winding 31 comprising five revolutions is arranged at the outer surface of the nose device. The distance between each screw windings is about 2 mm. The screw winding comprises ridges arranged in a spiral manner. Each ridge extends substantially perpendicular to the longitudinal axis of the nose device. The ridges are attached to the surface of the nose device at the base. The ridges may have a substantially rectangular cross-section as shown in FIG. 3. The ridges are arranged integrally with the nose device.

In another embodiment, the windings are made from another material, such as silver. The windings are arranged in a spiral at the outside of the nose device. Silver is known to have a bactericidal action.

The nose device is made from a transparent plastic material, such as polycarbonate.

The screw winding is arranged centrally at the nose device. The distance of the screw winding 31 from the distal end end is substantially the same as the distance of the screw winding from the proximal end of the nose device. Substantially the same may mean that the distance between the proximal end and the part of the screw winding being closest to the proximal end is between 0.3 and 3 times the distance between the distal end and the part of the screw winding being closest to the distal end. Additional screw threads may be arranged at the nose device for other purposes as discussed below.

The outer portion of the nose channel is normally not entirely cylindrical, but the partition between the nose channels results in a slightly distorted shape. The nose device 40 according to the embodiment shown in FIG. 4 is provided with a recess 45 intended to abut towards the partition when the nose device is in place. The recess 45 is adapted with a specific orientation in relation to the tab 42 so that the person using the nose device can adapt the nose device so that the recess 45 arranged opposite the nose partition. The tab 42 can be provided at the same angular position as the recess 45 or 180° in relation to the recess 45.

As shown in FIG. 5, the the ridges of the screw windings 51 can be arranged at an angle in relation to the longitudinal axis of the nose device. The distance or the pitch between the ridges of the screw windings is larger in comparison with the embodiment according to FIG. 3, for example 4 mm.

In the embodiment of FIG. 5, the ridges of the screw windings have a rectangular shape, wherein the direction of the ridges has an obtuse angle in relation to the longitudinal axis, i.e. an angle which is larger than 90°, as shown in FIG. 5. In this way, the nose device is prevented somewhat from being inserted in the nose channel and when it is rotated, the screw windings will be more gentle towards the cooperating channel walls.

As shown in FIG. 6, the ridges of the screw windings 61 may have an acute angle in relation to the longitudinal axis, i.e. an angle which is smaller than 90°, as shown in FIG. 6. In this case, the screw windings give a better screwing action at the inner surface of the nose channel walls, which is an advantage if a secure grip is desired.

FIG. 7 shows another embodiment of the nose device, in which the screw windings 71 are arranged with an outer rounded shape and with a distance between the windings of about 4 mm. Only three winding revolutions are used.

The distal end of the embodiment of FIG. 7 is provided with a cylindrical elongation 77 with an outer diameter, which is dimensioned for permitting passage of the distal end beyond the nose bone 5. A shoulder 78 cooperates with the nose bone 5 and prevents further movement of the nose device into the nose channel. The cylindrical elongation can be longer than is shown in FIG. 7 and continue in a flexible hose portion.

FIG. 8 shows the embodiment of FIG. 7 in which the cylindrical portion is replaced by an elastic device 86 adjacent the distal end of the nose device. The elastic device 86 is made from a soft, elastic and resilient material, such as polyurethane foam. The elastic device is intended to cooperate with the outer end of the nose bone 5 in order to form a shoulder towards the nose bone. In this way, the nose device will not be pushed further into the nose. When the nose device has been rotated, the elastic device will in addition exert a pressure or a force directed outwards, so that the sidewalls of the nose channel will be kept in a stretched position.

FIG. 9 shows the same embodiment as FIG. 1 and in addition provided with a longitudinal slit 97. The width of the slit may be adjusted by a distance member 98 arranged at the inside of the nose device, see FIG. 10. Thus, the diameter of the nose device may be adjusted.

As is shown in FIG. 10, the nose device is conical with a circular base. However, the base may be somewhat elliptical in order to suit the shape of the nose channel.

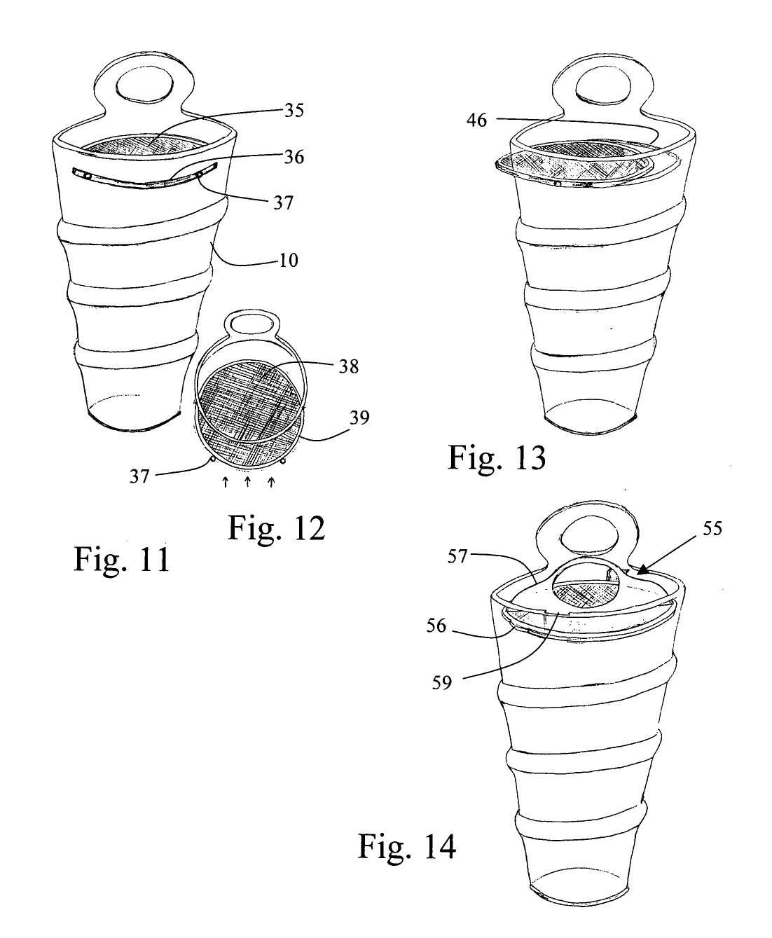

FIGS. 11 and 12 show a nose device comprising a filter 35, which is insertable in a slit 36 made in the nose expander at its outer end positioned closest to the nostril. As appears from FIG. 12, the filter comprises a circular frame 39, which retains and stretches a net or filter 38. Moreover, the filter is provided with two grip devices 37, which are used when the filter is inserted into or removed from the slit 36.

The slit 36 extends along 180° or slightly less of the periphery of the nose expander and is continued as an inner groove 46 as shown in FIG. 13. When the filter is inserted in the slit 36, it will pass there into with a certain friction and/or with a snap function and finally be present in the groove 46, possibly with a certain press-fit, so that the filter is retained in place.

The filter 38 comprises meshes with a suitable size for filtering the air, which is inhaled during breathing. The filter may be adapted to the application area. If the filter is to protect against pollen in the air, the filter should be arranged so that the filter collects the pollen. If the filter is to protect against bacteria in the air, the filter should have a corresponding dimensioning.

FIG. 14 shows another embodiment of the attachment of the filter to the nose device or nose expander. The filter 55 comprises a handle 57 and a bayonet connection comprising two extensions 56 positioned diametrically opposite to each other. The filter rests on an inner shoulder 58 as appears more clearly from FIG. 16. The shoulders 56 of the bayonet connection are insertable in two longitudinal recesses 59 as shown in FIGS. 14 and 16 and are rotated after the insertion over about 20° in order to be locked in a groove arranged at the inner side of the nose device. The filter is removable, for example with the reverse operation, for cleaning and reuse, or for exchange for another filter.

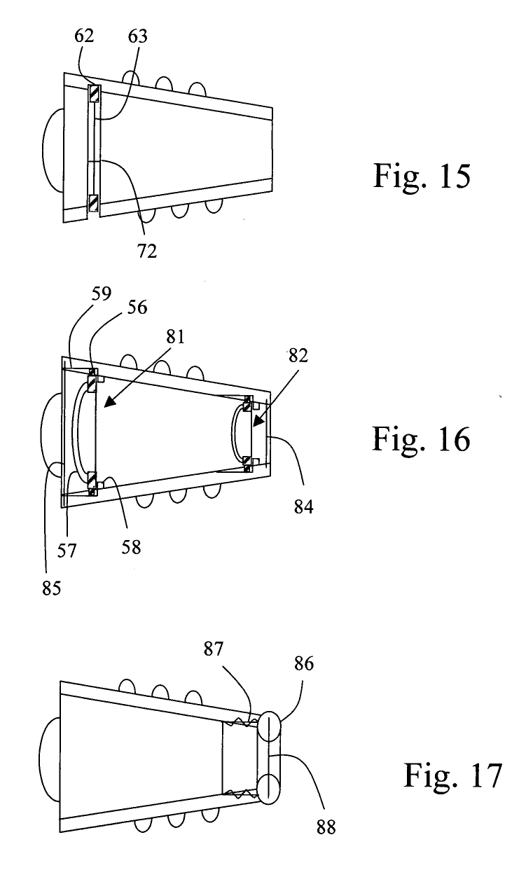

The construction of the filter appears from FIG. 15. The filter comprises a relatively stiff, circular frame 62, which supports and stretches a thin net 63. The frame 62 supports the net to make the assembly rigid. The net can be made from a suitable material and with suitable mesh sizes for the intended application. The net can be assembled centrally within the frame, as shown in FIG. 15, or be positioned adjacent an edge of the frame, as shown in FIG. 16.

FIG. 15 shows the embodiment of FIG. 11 or FIG. 13 in cross-section. The slit 36 extends over about 160° as shown by the line 72. In this way, the frame is insertable in the slit 36 with a press-fit, and is partly snapped into the groove 46.

FIG. 16 shows the use of two filters 81 and 82. The outer filter 81 may be intended for removing the largest contaminants, while the inner filter 82 can be a fine filter, with smaller mesh sizes. The outer filter 81 is reachable from the outside without removing the nose device from the nose and can easily be removed and cleaned at site. In order to exchange the filter 82, the nose device needs to be removed from the nostril, whereupon the filter 81 is removed in order to make the filter 82 accessible.

In an alternative embodiment, a filter 84 is integrally arranged at the inner end of the nose device as shown by line 84. When the integral filter 84 has been blocked, the entire nose device is discarded and a new nose device is applied. An integral filter 85 can alternatively be arranged at the outer end of the nose device, as an alternative to the filters 81 and 82.

The filter 81 is attached with a bayonet connection, but other connection methods can be used, such as screw winding or a snap connection.

FIG. 17 shows a filter with a screw winding 87, in which the filter is screwed to the inner end of the nose device. The filter 88 is integrated in an elastic device 86, the purpose of which is to protect the mucosa at the inner end. The filter may be combined with a filter 85 at the outer end, or with other types of removable filters. The filter 88 with screw winding can alternatively by arranged at the outer end.

The handle 57 can be arranged to comprise an attachment device for medical equipment, such as an oxygen supply device or a device for the administration of a medical agent, such as asthma medicine. A device for emitting a pleasant smell or other fragrances can also be attached to the handle 57, or be integrated into the nose device in another manner.

The nose device can be made in different sizes. Thus, there may be a child dimension and several adult dimensions, such as small, medium or large adult dimensions or male and female dimensions.

The above-mentioned dimensions can be varied with large ranges in order to form a nose device, which fits noses of different sizes. The cone angle can be between 5° and 15°, such as between 8° and 12°, such as 9° or 10°.

The length of the nose device can be between 10 mm and 40 mm, such as between 12 mm and 35 mm, such as between 15 mm and 30 mm, for example 20 mm or 25 mm.

The nose device may be manufactured in a plastic material, which is compatible with human use, such as polycarbonate, polyurethane, polyvinylcloride. The nose device can be made in a sandwich construction comprising a cellulose material, such as cardboard, covered with a plastic material.

The windings can be made from the same material as the nose device. Alternatively, the windings can be made in a separate material, such as silver or cupper. Other portions of the nose device may comprise silver or cupper is so desired.

The nose device can be provided with a medically active agent, such as histamine or a bactericidal agent, such as penicillin. The agent can be arranged at the outer surface of the nose device or be integrated in the material for some release thereof.

The nose device can be constructed for single use.

The nose device can also be used as a connection device for medical purposes, such as breathing of oxygen gas or other gases. For this purpose, the front end of the nose device may be provided with a connection device, such as a Luer connector. Alternatively, there may be arranged a bayonet connection or a screw winding or a similar device. The connection device can be an attachment device for any type of device, such as an air filter or a bacteria filter. A device for administration of a medical agent, such as asthma medicine can be arranged at the connection device. A device for releasing a pleasant scent and other fragrance can also be attached to the connection device, or be integrated in other manners in the nose device.

In the drawings, there is shown a single winding, but the winding may comprise a double winding, for example with a winding distance of 2.5 mm and a pitch of 5 mm. Thus, it is sufficient with half a revolution for stretching the nose mucosa with 2.5 mm in the length direction. Alternatively, there may be a single winding with a pitch of about 2 mm. Then, a rotation over 1.5 revolutions results in a stretching of the nose mucosa with 3 mm.

In the claims, the expression comprises/comprising does not exclude the presence of other members or steps. Also, if they are defined individually, several devices, members or method steps can be performed by for example a single unit. Also, if individual features are included in the different claims and different embodiments, these can possibly be combined with advantage and the incorporation in different claims does not mean that a combination of features is not feasible and/or advantageous. A single reference does not exclude a plurality. The expressions “a” “an”, “first”, “second” and so on, does not exclude a plurality. Reference numerals in the claims are provided only as clarifying examples and shall not be regarded as limiting the scope of the claims in any way.

Although the present invention has been described above with reference to specific embodiments, the invention is not intended to be limited to specific forms defined. Rather, the invention is limited only by the appended claims and other embodiments than those given above are equally possible within the scope of the appended claims.

1. A nose device intended to be inserted into the nose channel of a person, comprising
a conical portion with a distal end intended to be adapted adjacent a nose bone and a proximal end intended to be arranged adjacent a nostril, whereby the distal end is the small end of the conical portion, characterized by
screw windings arranged centrally at the outer surface of the conical portion.

2. The nose device according to claim 1, characterized by
a tab portion arranged adjacent the proximal end of the conical device.

3. The nose device according to claim 1, characterized by
a recess arranged at a side surface of the conical portion.

4. Nose device according to claim 1, wherein
said screw windings comprise ridges having an essentially rectangular cross-sectional shape.

5. Nose device according to claim 1, characterized by
a filter device arranged covering the internal passage of the conical portion for filtering air passing through the conical portion.

6. The nose device according to claim 1, characterized by
an elastic device arranged at the distal end of the conical portion.

7. Nose device according to claim 1, characterized by
a medical agent adapted at the nose device, such as histamine or penicillin.

8. The nose device according to claim 1, characterized by
a connection device for attachment of a device, such as a gas supply device.

9. The nose device according to claim 1, characterized by
a cylindrical portion at the distal end comprising a shoulder for cooperation with a nose bone.

10. The nose device according to claim 1, wherein
the nose device is made in a single piece of a plastic material, such as polycarbonate, polyurethane or polyvinylcloride.
